(19) United States
(12) Patent Application Publication
(10) Pub. No.: US 2025/0101204 A1
(43) Pub. Date: Mar. 27, 2025

(54) COMPOSITIONS AND METHODS FOR PROTECTING AGAINST CELL DAMAGE AND INFLAMMATION

(71) Applicant: ARIZONA BOARD OF REGENTS ON BEHALF OF THE UNIVERSITY OF ARIZONA, Tucson, AZ (US)

(72) Inventors: Ruslan Rafikov, Tucson, AZ (US); Olga Rafikova, Tucson, AZ (US)

(21) Appl. No.: 18/725,609

(22) PCT Filed: Feb. 28, 2023
(86) PCT No.: PCT/US23/14153

§ 371 Date: Jun. 28, 2024

Related U.S. Application Data
(60) Provisional application No. 63/314,911, filed on Feb. 28, 2022.

(51) Int. Cl. C08K 7/08; A61K 38/10

(52) U.S. Cl. CPC C08K 7/08; A61K 38/10

(57) ABSTRACT

High mobility group box protein 1 (HMGB1) is a nuclear protein released during cell damage and stimulates inflammatory and proliferative pathways in the surviving neighboring cells. HMGB1 contains three cysteine residues (e.g., 23/45/106) involved in the extracellular HMGB1 oligomerization and receptor binding. The peptide compositions described herein shield the aforementioned cysteine residues, which block HMGB1 from binding to DNA and, thus, reduce apoptotic signaling within a cell. Furthermore, methods of treating inflammatory disease (e.g., PAH) by administering said peptide composition are described herein.

### CROSS-REFERENCES TO RELATED APPLICATIONS

This application claims benefit of U.S. Provisional Application No. 63/314,911 filed Feb. 28, 2022, the specification of which is incorporated herein in their entirety by reference.

### STATEMENT REGARDING FEDERALLY SPONSORED RESEARCH OR DEVELOPMENT

This invention was made with government support under Grant No. HL133085 and HL132918, awarded by National Institutes of Health. The government has certain rights in the invention.

### REFERENCE TO AN ELECTRONIC SEQUENCE LISTING

The contents of the electronic sequence listing (ARIZ_22_06_PCT_Sequence_Listing.xml; Size: 36,869 bytes; and Date of Creation: Feb. 24, 2023) is herein incorporated by reference in its entirety.

### FIELD OF THE INVENTION

The features compositions for peptide-based modulators of HMGBL activity to protect against cell damage and inflammation and methods for using said compositions.

### BACKGROUND OF THE INVENTION

The high mobility group protein B1 (HMGB1) is a nuclear chromatin-like protein that is a highly abundant protein with roles in several cellular processes, such as chromatin structure and transcriptional regulation and an extracellular role in inflammation. HMGB1 regulates the transcriptional activity of several targets. During the cell damage, HMGB1 also gets released outside the cell and mediates inflammatory response by activating TLR4 receptors (toll-like receptors) and triggering inflammatory cascades. HMGB1 contains three cysteine residues (23/45/106) involved in the extracellular HMGB1 oligomerization and receptor binding. However, the role of cysteines in intracellular HMGB1 activity is less established.

### BRIEF SUMMARY OF THE INVENTION

It is an objective of the present invention to provide compositions and methods that allow for peptide-based modulation of HMGBL activity to protect against cell damage and inflammation, as specified in the independent claims. Embodiments of the invention are given in the dependent claims. Embodiments of the present invention can be freely combined with each other if they are not mutually exclusive.

High mobility group box protein 1 (HMGB1) is a nuclear protein released during cell damage and stimulates inflammatory and proliferative pathways in the surviving neighboring cells. Additionally, HMGB1 is an important contributor to the pathogenesis of pulmonary arterial hypertension (PAH). Its dual activity includes alarmin properties initiated by HMGB1 release from damaged cells and nuclear activity, which involves binding to DNA and regulating the expression of specific genes.

The present invention describes peptide inhibitors designed to shield HMGB1 Cysteine 106, which is responsible for binding HMGB1 to DNA and TLR4. By decreasing HMGB1 binding to DNA, the inhibitory peptides enhance the exit of nuclear HMGB1 to the cytosol, where it undergoes proteasomal degradation. Furthermore, the reduced HMGB1/DNA binding negatively affects the expression of pro-apoptotic p53 and pro-inflammatory TLR4. In the animal model of pulmonary arterial hypertension (PAH), the inhibitory peptides produce an anti-apoptotic effect in the lungs and heart and reduce the levels of circulating HMGB1 released by dying cells. These changes correspond with a decrease in PAH severity when the treatment was applied at the early stage of PAH and protected the right ventricle (RV) function at the later stage. Thus, the peptides represent the unique therapy that could either treat the disease or protect the heart from failing at the advanced stages. Given that RV dysfunction is the primary cause of death in patients with PAH, this therapy could decrease patient mortality. The protective effect is especially evident in females, predominantly affected by PAH. The anti-apoptotic effect and reduced inflammatory response induced by anti-HMGB1 peptide inhibitors are also important therapeutic goals in a large spectrum of other diseases, including but not limited to diabetic complications, hypertension, systemic inflammation, acute and chronic heart, kidney, liver diseases and/or cancer, e.g., bladder cancer, breast cancer, lung cancer, prostate cancer, cervical cancer, colorectal cancer, kidney cancer, melanoma, non-Hodgkin lymphoma, leukemia, endometrial cancer, pancreatic cancer, thyroid cancer, liver cancer, etc.

In some embodiments, the present invention features an anti-apoptotic peptide comprising a sequence at least 80% identical to FFLFCSEYRPKIK (SEQ ID NO: 1) or SIGDVAKKLGEMWNN (SEQ ID NO: 2), wherein the peptide binds to a high mobility group box 1 (HMGB1) protein.

One of the unique and inventive technical features of the present invention is a peptide designed to shield a cysteine of HMGB1 (e.g., either Cys106 or Cys23/45). Without wishing to limit the invention to any theory or mechanism, it is believed that the technical feature of the present invention advantageously provides for the HMGB1 protein to avoid interaction with cellular targets, reduce the ability of HMGB1 to bind to DNA, increases the exit of nuclear HMGB1 to the cytosol and its subsequent degradation by proteasomes. None of the presently known prior references or work has the unique, inventive technical feature of the present invention.

Furthermore, the prior references teach away from the present invention. For example, it was unclear whether peptides could mediate the action via binding to DNA, leading to decreased HMGB1/DNA interaction.

Furthermore, the inventive technical features of the present invention contributed to a surprising result. For example, it was not expected that peptides could induce proteasomal degradation of HMGB1, thus decreasing HMGB1 availability for inflammatory response.

Any feature or combination of features described herein are within the scope of the present invention provided that the features included in any such combination are not mutually inconsistent as will be apparent from the context, this specification, and the knowledge of one of ordinary skill in the art. Additional advantages and aspects of the invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 1C, and 1D show only intracellular Cysteine¹⁰⁶ lowers HMGB1 levels in both fractions. FIGS. 1A and 1B show peptides that bind to HMGB1^{Cys106} but not HMGB1^{Cys23/45} reduce HMGB1 levels in PASMC cytosolic and nuclear fraction. FIGS. 1C and 1D show only the HMGB1^{Cys106} peptide that enters the cell reduces HMGB1 levels in PASMC cytosolic and nuclear fraction.

FIGS. 2A and 2B show Cysteine¹⁰⁰ increases HMGB1 expulsion from the nucleus to cytosol. A proteasome inhibitor (e.g., MG 132) restores cytosolic (FIG. 2A) and nuclear (FIG. 2B) HMGB1 levels reduced by αHMGB1^{Cys106} peptide.

FIG. 3 shows the αHMGB1^{Cys106} peptide (e.g., SEQ. ID NO: 4) prevents HMGB1 binding to DNA. The left two gel blots show HMGB1 elution after incubation with DNA cellulose.

FIG. 4 shows intracellular HMGB1^{Cys106} peptide (e.g., SEQ. ID NO: 4) reduces p53 levels in nuclei of PASMC.

FIGS. 5A, 5B, and 5C show the αHMGB1^{Cys106} peptide (e.g., SEQ. ID NO: 4) provides protective and treatment effects in rat Su/Hx model of PAH.

FIG. 6 shows intracellular HMGB1^{Cys106} peptide (e.g., SEQ. ID NO: 4) reduces cytosolic and nuclear TLR4 protein levels.

FIG. 7 shows the αHMGB1^{Cys106} peptide (e.g., SEQ. ID NO: 4) attenuates apoptosis induced by the p53 activator.

FIG. 8 shows the αHMGB1^{Cys106} peptide (e.g., SEQ. ID NO: 4) decreases apoptosis in the lungs of Su/Hx female rats.

FIG. 9 shows the anti-fibrotic activity of αHMGB1^{Cys106} peptide (e.g., SEQ. ID NO: 4) in the heart of male Su/Hx rats. Female rats do not develop cardiac fibrosis.

FIG. 10 shows sex differences in HMGB1 cellular distribution in response to αHMGB1^{Cys106} peptide (e.g., SEQ. ID NO: 4).

FIGS. 11A and 11B show the sex differences in plasma HMGB1 levels in response to αHMGB1^{Cys106} peptides (e.g., SEQ. ID NO: 4) in early PH stages. Note: SU2 is related to the 2 weeks after exposure to SU5416 (PAH inducer), and is indicative of a relatively early disease stage; SU5 means 5 weeks after exposure to SU5416, and is indicative of an advanced stage of the disease.

FIG. 12 shows the protective effect of αHMGB1^{Cys106} peptide (e.g., SEQ. ID NO: 4) against cardiomyocyte hypertrophy.

FIG. 13A shows the αHMGB1^{Cys106} peptide (e.g., SEQ. ID NO: 4) reduces expression of p53 in vivo.

FIG. 13B shows the αHMGB1^{Cys106} peptide (e.g., SEQ. ID NO: 4) mediates protection against apoptosis in female but not male lungs.

FIG. 13C shows the αHMGB1^{Cys106} peptide (e.g., SEQ. ID NO: 4) protects against senescence in female and early-stage male PAH.

FIG. 13D shows the αHMGB1^{Cys106} peptide (e.g., SEQ. ID NO: 4) protects against necroptosis in males at early PAH.

FIG. 13E shows the αHMGB1^{Cys106} peptide (e.g., SEQ. ID NO: 4) protects against genotoxic stress.

FIG. 14A shows the anti-apoptotic effect of the αHMGB1^{Cys106} peptide (e.g., SEQ. ID NO: 4) in human pulmonary artery endothelial cells (HPAEC) stimulated by p53 against RITA.

FIG. 14B shows the protective effect of the αHMGB1^{Cys106} peptide (e.g., SEQ. ID NO: 4) against genotoxic stress induced in human pulmonary artery endothelial cells (HPAEC) by Doxorubicin (1 μg/ml for 24 h).

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the terms “subject” and “patient” are used interchangeably. As used herein, a subject can be an animal (amphibian, reptile, avian, fish, or mammal) such as a non-primate (e.g., cows, pigs, horses, cats, dogs, rats, etc.) or a primate (e.g., monkey, ape, and human). In specific embodiments, the subject is a human. In one embodiment, the subject is a mammal (e.g., a human) having a disease, disorder, or condition described herein. In another embodiment, the subject is a mammal (e.g., a human) at risk of developing a disease, disorder, or condition described herein. In certain instances, the term patient refers to a human under medical care.

The terms “treating” or “treatment” refer to any indicia of success or amelioration of the progression, severity, and/or duration of a disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread, or worsening of a disease or disorder or of one or more symptoms thereof. In certain cases, the beneficial effects that a subject derives from a prophylactic or therapeutic agent do not result in a cure of the disease or disorder.

The term “effective amount” as used herein refers to the amount of a therapy or medication which is sufficient to reduce and/or ameliorate the severity and/or duration of a given disease, disorder, or condition and/or a symptom related thereto. This term also encompasses an amount necessary for the reduction or amelioration of the advancement or progression of a given disease (e.g., PAH), disorder or condition, reduction or amelioration of the recurrence, development or onset of a given disease, disorder or condition, and/or to improve or enhance the prophylactic or therapeutic effect(s) of another therapy. In some embodiments, “effective amount,” as used herein also refers to the amount of therapy provided herein to achieve a specified result.

As used herein, and unless otherwise specified, the term “therapeutically effective amount” is an amount sufficient enough to provide a therapeutic benefit in the treatment or management of a disease or to delay or minimize one or more symptoms associated with the presence of the cardiovascular disease. A therapeutically effective amount of an agent means an amount of therapeutic agent, alone or in combination with other therapies, which provides a therapeutic benefit in the treatment or management of the disease. The term “therapeutically effective amount” can encompass an amount that improves overall therapy, reduces or avoids symptoms or causes of cardiovascular disease, or enhances the therapeutic efficacy of another therapeutic agent.

The terms “administering” and “administration” refer to methods of providing a pharmaceutical preparation to a subject. Such methods are well known to those skilled in the art and include, but are not limited to, administering the compositions orally, intranasally, parenterally (e.g., intravenously and subcutaneously), by intramuscular injection, by intraperitoneal injection, intrathecally, transdermally, extracorporeally, topically or the like.

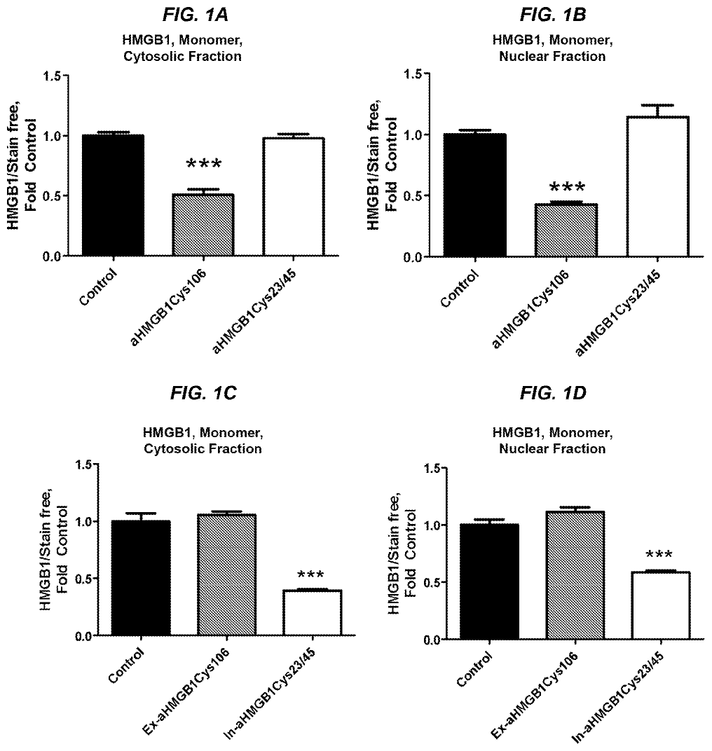

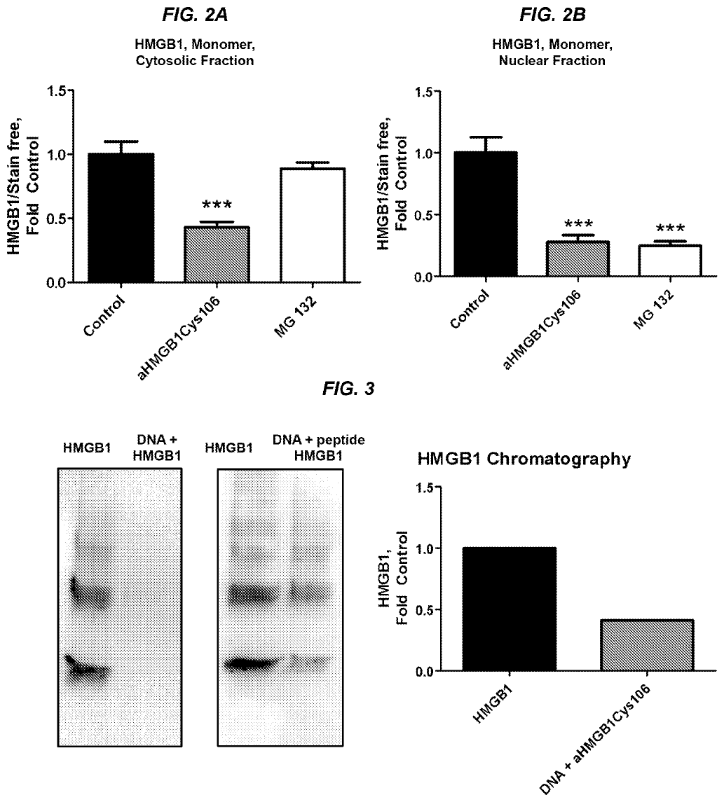

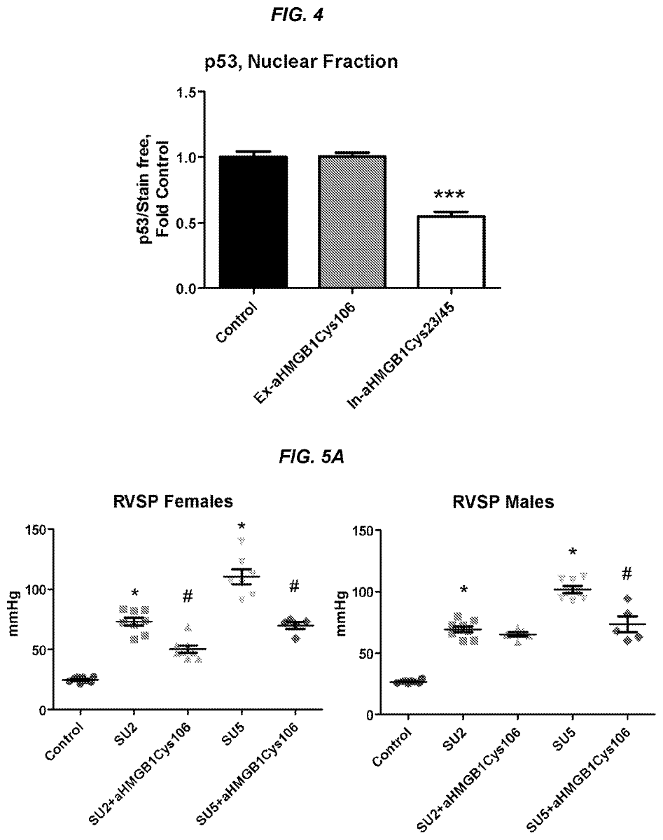

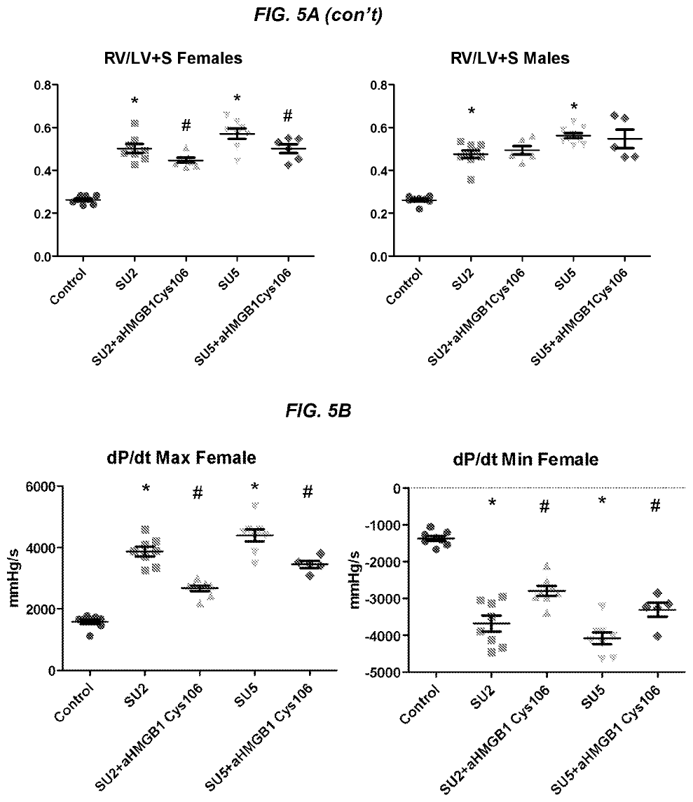

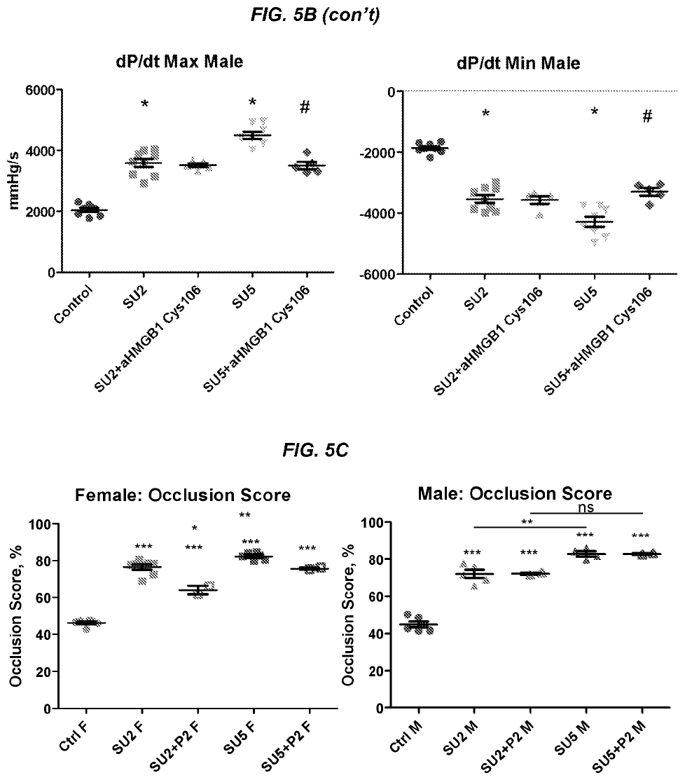

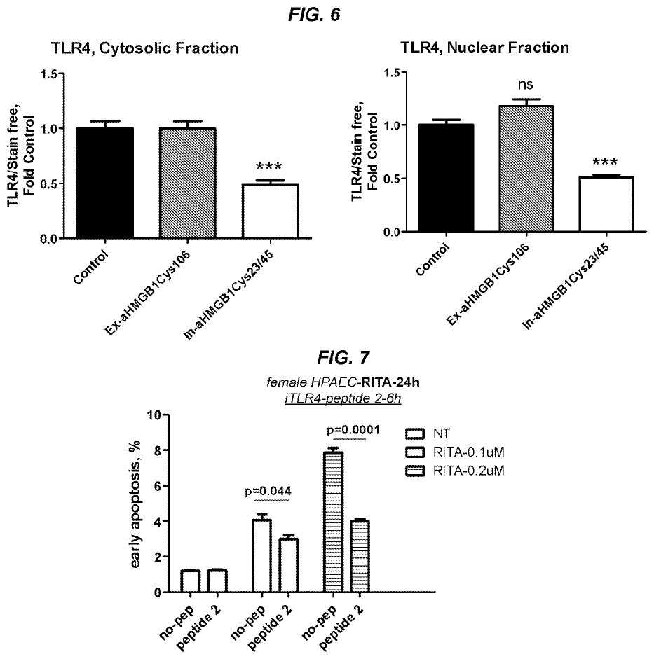

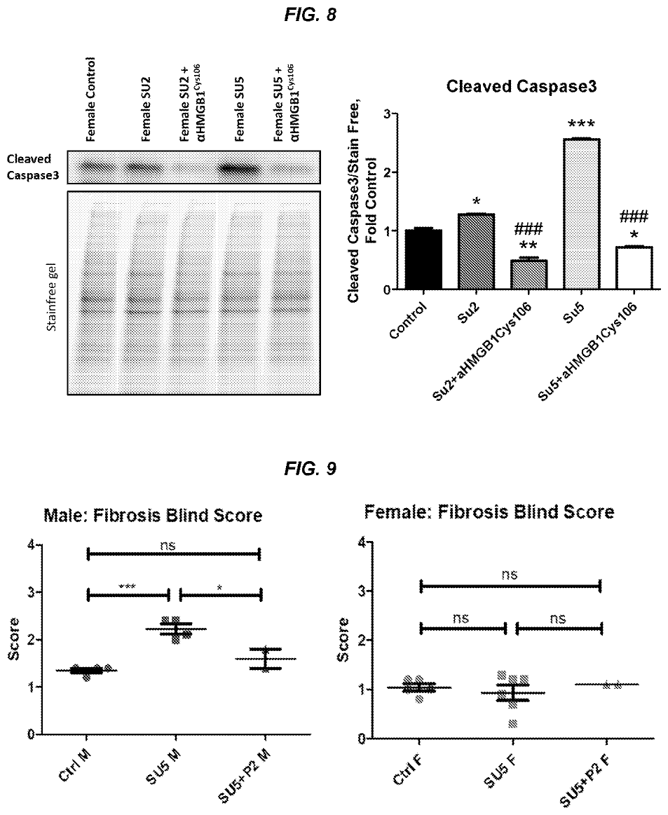

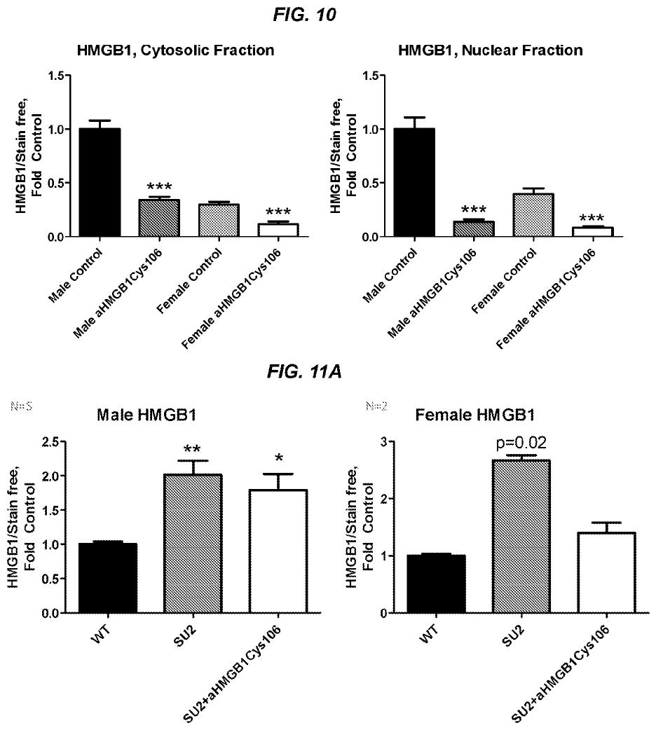

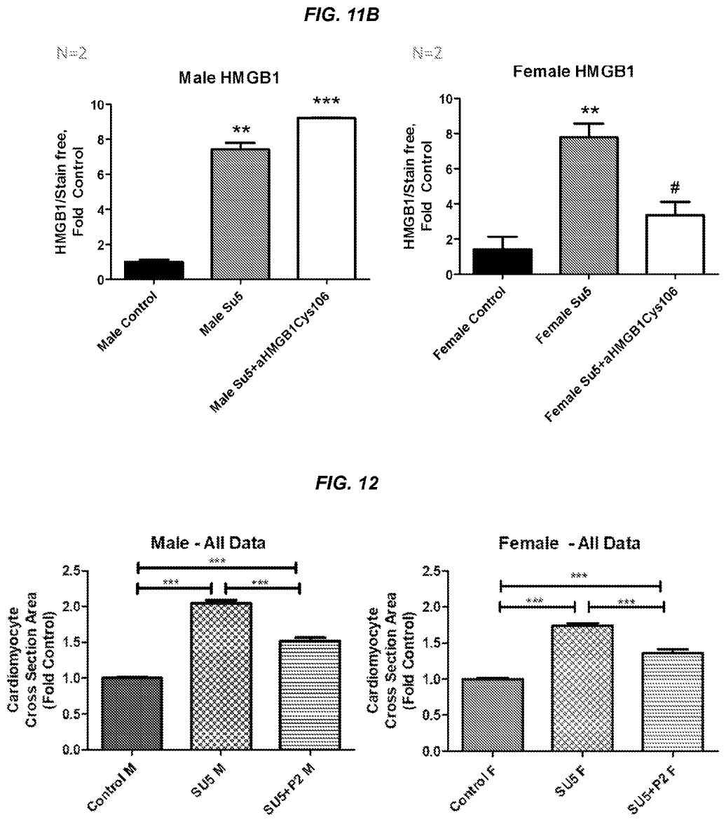

Referring now to FIGS. 1A-14B, the present invention features a peptide (e.g., an anti-apoptotic) comprising a sequence at least 84% identical to FFLFCSEYRPKIK (SEQ ID NO: 1) or SIGDVAKKLGEMWNN (SEQ ID NO: 2). In some embodiments, the peptide binds to a high mobility group box 1 (HMGB1) protein.

In some embodiments, the peptides described herein comprise a sequence at least 53% identical to FFLFCSEYRPKIK (SEQ ID NO: 1). In some embodiments, the peptides described herein comprise a sequence at least 61% identical to FFLFCSEYRPKIK (SEQ ID NO: 1). In some embodiments, the peptides described herein comprise a sequence at least 69% identical to FFLFCSEYRPKIK (SEQ ID NO: 1). In some embodiments, the peptides described herein comprise a sequence at least 76% identical to FFLFCSEYRPKIK (SEQ ID NO: 1). In some embodiments, the peptides described herein comprise a sequence at least 84% identical to FFLFCSEYRPKIK (SEQ ID NO: 1). In some embodiments, the peptides described herein comprise a sequence at least 92% identical to FFLFCSEYRPKIK (SEQ ID NO: 1). In some embodiments, the peptides described herein comprise a sequence 100% identical to FFLFCSEYRPKIK (SEQ ID NO: 1). In some embodiments, the aforementioned peptides (e.g., SEQ ID NO: 1) bind to a specific region of HMGB1 and shield Cys106.

In some embodiments, the peptides described herein comprise a sequence at least 53% identical to SIGDVAKKLGEMWNN (SEQ ID NO: 2). In some embodiments, the peptides described herein comprise a sequence at least 60% identical to SIGDVAKKLGEMWNN (SEQ ID NO: 2). In some embodiments, the peptides described herein comprise a sequence at least 66% identical to SIGDVAKKLGEMWNN (SEQ ID NO: 2). In some embodiments, the peptides described herein comprise a sequence at least 73% identical to SIGDVAKKLGEMWNN (SEQ ID NO: 2). In some embodiments, the peptides described herein comprise a sequence at least 80% identical to SIGDVAKKLGEMWNN (SEQ ID NO: 2). In some embodiments, the peptides described herein comprise a sequence at least 86% identical to SIGDVAKKLGEMWNN (SEQ ID NO: 2). In some embodiments, the peptides described herein comprise a sequence at least 93% identical to SIGDVAKKLGEMWNN (SEQ ID NO: 2). In some embodiments, the peptides described herein comprise a sequence 100% identical to SIGDVAKKLGEMWNN (SEQ ID NO: 2). In some embodiments, the aforementioned peptides (e.g., SEQ ID NO: 2) bind to a specific region of HMGB1 and shield Cys23/45.

In some embodiments, the peptides described herein comprise at least one modification. In other embodiments, the peptides described herein comprise at least two modifications. In further embodiments, the peptides described herein comprise at least three modifications. In some embodiments, the modification is a substitution. In some embodiments, the modification is a substitution or a deletion.

In some embodiments, at least one of the F amino acids is substituted with a Y or a W amino acid. In some embodiments, the L amino acid is substituted with an I or a V amino acid. In some embodiments, the C amino acid is substituted with an M amino acid. In some embodiments, the S amino acid is substituted with a C or an M amino acid. In some embodiments, the Y amino acid is substituted with an F or a W amino acid. In some embodiments, the R amino acid is substituted with a K amino acid. In some embodiments, at least one of the K amino acids is substituted with an R amino acid. In some embodiments, the I amino acid is substituted with an L or a V amino acid. In some embodiments, at least one of the G amino acids is substituted with an A amino acid. In some embodiments, the A amino acid is substituted with a G amino acid. In some embodiments, the D amino acid is substituted with an E amino acid. In some embodiments, the E amino acid is substituted with a D amino acid. In some embodiments, the V amino acid is substituted with an L or an I amino acid. In some embodiments, the M amino acid is substituted with an S or a C amino acid. In some embodiments, at least one of the N amino acids is substituted with a Q amino acid.

TABLE 1
Non-limiting examples of a peptide comprising a
singular substitution modification:

| SEQ | | SEQ | |
|---|---|---|---|
| ID NO: | Sequence | ID NO: | Sequence |
| 6 | YFLFCSEYRPKIK | 24 | FFLFCSEYRPRIK |
| 7 | FYLFCSEYRPKIK | 25 | FFLFCSEYRPKIR |
| 8 | FFLYCSEYRPKIK | 26 | FFLFCSEYRPKLK |
| 9 | WFLFCSEYRPKIK | 27 | FFLFCSEYRPKVK |
| 10 | FWLFCSEYRPKIK | 28 | SLGDVAKKLGEMWNN |
| 11 | FFLWCSEYRPKIK | 29 | SVGDVAKKLGEMWNN |
| 12 | FFIFCSEYRPKIK | 30 | SIADVAKKLGEMWNN |
| 13 | FFVFCSEYRPKIK | 31 | SIGDVAKKLAEMWNN |
| 14 | SIGDVAKKIGEMWNN | 32 | SIGDVGKKLGEMWNN |
| 15 | SIGDVAKKVGEMWNN | 33 | SIGEVAKKLGEMWNN |
| 16 | FFLFMSEYRPKIK | 34 | FFLFCSDYRPKIK |
| 17 | FFLFCCEYRPKIK | 35 | SIGDVAKKLGDMWNN |
| 18 | FFLFMSEYRPKIK | 36 | SIGDLAKKLGEMWNN |
| 19 | CIGDVAKKLGEMWNN | 37 | SIGDIAKKLGEMWNN |
| 20 | MIGDVAKKLGEMWNN | 38 | SIGDVAKKLGESWNN |
| 21 | FFLFCSEFRPKIK | 39 | SIGDVAKKLGECWNN |
| 22 | FFLFCSEWRPKIK | 40 | SIGDVAKKLGEMWQN |
| 23 | FFLFCSEYKPKIK | 41 | SIGDVAKKLGEMWNQ |

In other embodiments, the peptide is selected from a group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

In some embodiments, the peptides described herein comprise a sequence at least 60% identical to the aforementioned sequences. In some embodiments, the peptides described herein comprise a sequence at least 75% identical to the aforementioned sequences. In some embodiments, the peptides described herein comprise a sequence at least 85% identical to the aforementioned sequences. In some embodiments, the peptides described herein comprise a sequence at least 90% identical to the aforementioned sequences. In some embodiments, the peptides described herein comprise a sequence at least 100% identical to the aforementioned sequences.

In some embodiments, the peptides described herein further comprise a cell membrane crossing sequence. In some embodiments, the cell membrane crossing sequence comprises GRKKRRQRRRPQ (SEQ ID NO: 3). In some embodiments, the cell membrane crossing sequence comprises a sequence at least 92% identical to SEQ ID NO: 3. In some embodiments, the cell membrane crossing sequence comprises a sequence at least 83% identical to SEQ ID NO: 3. In some embodiments, the cell membrane crossing sequence comprises a sequence at least 75% identical to SEQ ID NO: 3. In some embodiments, the cell membrane crossing sequence comprises a sequence at least 67% identical to SEQ ID NO: 3. Other cell membrane crossing sequences (i.e., cell membrane penetrating sequence) may be used in accordance with the present invention.

In some embodiments, the cell membrane crossing sequence is modified. In some embodiments, the cell membrane crossing sequence is modified with a chemical group. In some embodiments, the chemical group is an amidation. In some embodiments, the cell membrane crossing sequence comprises modified amino acids (e.g., phosphorylated). In some embodiments, the chemical group is a triphenylphosphonium group or derivative thereof. In some embodiments, the membrane crossing sequence is shortened. For example, the membrane crossing sequence is shortened by one amino acid, or two amino acids, or three amino acids. In some embodiments, the membrane crossing sequence (e.g., SEQ ID NO: 3) is shortened by two or three positively charged amino acids (e.g., R, K, H).

In other embodiments, the membrane crossing sequence comprises extra amino acid residues. In some embodiments, the membrane crossing sequence comprises one extra amino acid residue. In some embodiments, the membrane crossing sequence comprises two extra amino acid residues. In some embodiments, the membrane crossing sequence comprises three extra amino acid residues. In some embodiments, the membrane crossing sequence comprises five extra amino acid residues. The crossing membrane sequence may comprise more than five, ten, or twenty extra amino acid residues.

In some embodiments, the peptide comprises a sequence at least 80% identical to

(SEQ ID NO: 4)
FFLFCSEYRPKIKGRKKRRQRRRPQ
or
(SEQ ID NO: 5)
SIGDVAKKLGEMWNNGRKKRRQRRRPQ.

The present invention may feature a peptide (e.g., an anti-apoptotic peptide) comprising a sequence at least 80% identical to FFLFCSEYRPKIKGRKKRRQRRRPQ (SEQ ID NO: 4) or SIGDVAKKLGEMWNNGRKKRRQRRRPQ (SEQ ID NO: 5). In some embodiments, the aforementioned peptides (e.g., SEQ ID NO: 4 or SEQ ID NO: 5) bind to a high mobility group box 1 (HMGB1) protein.

In some embodiments, the peptides described herein comprise a sequence at least 96% identical to FFLFCSEYRPKIKGRKKRRQRRRPQ (SEQ ID NO: 4). In some embodiments, the peptides described herein comprise a sequence at least 92% identical to FFLFCSEYRPKIKGRKKRRQRRRPQ (SEQ ID NO: 4). In some embodiments, the peptides described herein comprise a sequence at least 88% identical to FFLFCSEYRPKIKGRKKRRQRRRPQ (SEQ ID NO: 4). In some embodiments, the peptides described herein comprise a sequence at least 84% identical to FFLFCSEYRPKIKGRKKRRQRRRPQ (SEQ ID NO: 4). In some embodiments, the peptides described herein comprise a sequence at least 80% identical to FFLFCSEYRPKIKGRKKRRQRRRPQ (SEQ ID NO: 4). In some embodiments, the peptides described herein comprise a sequence at least 76% identical to FFLFCSEYRPKIKGRKKRRQRRRPQ (SEQ ID NO: 4). In some embodiments, the peptides described herein comprise a sequence at least 72% identical to FFLFCSEYRPKIKGRKKRRQRRRPQ (SEQ ID NO: 4). In some embodiments, the peptides described herein comprise a sequence at least 60% identical to FFLFCSEYRPKIKGRKKRRQRRRPQ (SEQ ID NO: 4).

In some embodiments, the peptides described herein comprise a sequence at least 96% identical to SIGDVAKKLGEMWNNGRKKRRQRRRPQ (SEQ ID NO: 5). In some embodiments, the peptides described herein comprise a sequence at least 93% identical to SIGDVAKKLGEMWNNGRKKRRQRRRPQ (SEQ ID NO: 5). In some embodiments, the peptides described herein comprise a sequence at least 89% identical to SIGDVAKKLGEMWNNGRKKRRQRRRPQ (SEQ ID NO: 5). In some embodiments, the peptides described herein comprise a sequence at least 85% identical to SIGDVAKKLGEMWNNGRKKRRQRRRPQ (SEQ ID NO: 5). In some embodiments, the peptides described herein comprise a sequence at least 81% identical to SIGDVAKKLGEMWNNGRKKRRQRRRPQ (SEQ ID NO: 5). In some embodiments, the peptides described herein comprise a sequence at least 78% identical to SIGDVAKKLGEMWNNGRKKRRQRRRPQ (SEQ ID NO: 5). In some embodiments, the peptides described herein comprise a sequence at least 74% identical to SIGDVAKKLGEMWNNGRKKRRQRRRPQ (SEQ ID NO: 5). In some embodiments, the peptides described herein comprise a sequence at least 70% identical to SIGDVAKKLGEMWNNGRKKRRQRRRPQ (SEQ ID NO: 5). In some embodiments, the peptides described herein comprise a sequence at least 63% identical to SIGDVAKKLGEMWNNGRKKRRQRRRPQ (SEQ ID NO: 5).

In some embodiments, the amino acids are D-amino acids, L-amino acids, or combinations thereof. In some embodiments, the amino acids are unnatural amino acids, modified amino acids (e.g., phosphorylated), or a combination thereof. In some embodiments, the N-terminal or the C-terminal of the peptide described herein is modified. In some embodiments, the modification comprises adding a chemical moiety, a membrane crossing sequence, or a chemical group to the N-terminal or the C-terminal. Non-limiting examples of modifications include but are not limited to a triphenylphosphonium group, a TAT sequence, polyimide or polyethylene glycol, or other hydrophobic and positively charged chemical groups. In some embodiments, the modification is a TAT sequence or a triphenylphosphonium group.

Without wishing to limit the present invention to any theories or mechanisms, it is believed that the peptides described herein shield the HMGB1^{Cys106} and prevent its interaction with cellular targets, reduce the ability of HMGB1 to bind to DNA, increase the exit of nuclear HMGB1 to the cytosol and its subsequent degradation by proteasomes. Decreased HMGB1/DNA binding negatively affects the expression of TLR4 and p53. In females, the anti-apoptotic effect of αHMGB1^{Cys106} peptide decreases right ventricle (RV) systolic pressure at the early stage of PAH and protects the RV at the later stage. In contrast, males show an elevated HMGB1 expression and no apoptosis activation in response to PAH. Therefore, the anti-apoptotic effect of αHMGB1^{Cys106} peptide does not manifest in males.

In some embodiments, the peptide binds at or around Cys106 or Cys23/45 of the HMGB1 protein. In some embodiments, the peptide inhibits the HMGB1 interaction with DNA.

The present invention features a method of treating pulmonary arterial hypertension (PAH) in a subject in need thereof. The method may comprise administering a therapeutically effective amount of a peptide as described herein to the subject. In some embodiments, the subject is female.

The present invention may also feature a method of treating an inflammatory disease in a subject in need thereof. The method may comprise administering a therapeutically effective amount of a peptide as described herein to the subject. In some embodiments, the inflammatory disease is hypertension, systemic inflammation, acute or chronic heart disease, kidney disease, or liver disease.

In some embodiments, the peptides described herein have an anti-apoptotic effect and reduce the inflammatory response. In some embodiments, the peptides described herein may treat diabetic complications, hypertension, systemic inflammation, acute and chronic heart, kidney, liver diseases and/or cancer, e.g., bladder cancer, breast cancer, lung cancer, prostate cancer, cervical cancer, colorectal cancer, kidney cancer, melanoma, non-Hodgkin lymphoma, leukemia, endometrial cancer, pancreatic cancer, thyroid cancer, liver cancer, etc.

The present invention also features a method of inhibiting a high mobility group box 1 (HMGB1) protein interaction with DNA in vitro. The method may comprise administering a therapeutically effective amount of a peptide as described herein to a cell or an in vitro system. The present invention may also feature a method of inhibiting a high mobility group box 1 (HMGB1) protein interaction with DNA in a subject. The method may comprise administering a peptide as described herein to the subject.

The present invention may further feature a peptide (e.g., an anti-apoptotic peptide) comprising a sequence at least 84% identical to FFLFCSEYRPKIK (SEQ ID NO: 1) or SIGDVAKKLGEMWNN (SEQ ID NO: 2) for use in a method for treatment of an inflammatory disease. In some embodiments, the inflammatory disease is hypertension, systemic inflammation, acute or chronic heart disease, kidney disease, or liver disease.

The present invention may feature a peptide (e.g., an anti-apoptotic peptide) comprising a sequence at least 80% identical to FFLFCSEYRPKIKGRKKRRQRRRPQ (SEQ ID NO: 4) or SIGDVAKKLGEMWNNGRKKRRQRRRPQ (SEQ ID NO: 5) for use in a method for treatment of inflammatory disease. In some embodiments, the inflammatory disease is hypertension, systemic inflammation, acute or chronic heart disease, kidney disease, or liver disease.

The present invention may also feature a peptide (e.g., an anti-apoptotic peptide) comprising a sequence at least 84% identical to FFLFCSEYRPKIK (SEQ ID NO: 1) or SIGDVAKKLGEMWNN (SEQ ID NO: 2) for use in a method for treatment of pulmonary arterial hypertension (PAH).

The present invention may feature a peptide (e.g., an anti-apoptotic peptide) comprising a sequence at least 80% identical to FFLFCSEYRPKIKGRKKRRQRRRPQ (SEQ ID NO: 4) or SIGDVAKKLGEMWNNGRKKRRQRRRPQ (SEQ ID NO: 5) for use in a method for treatment of PAH.

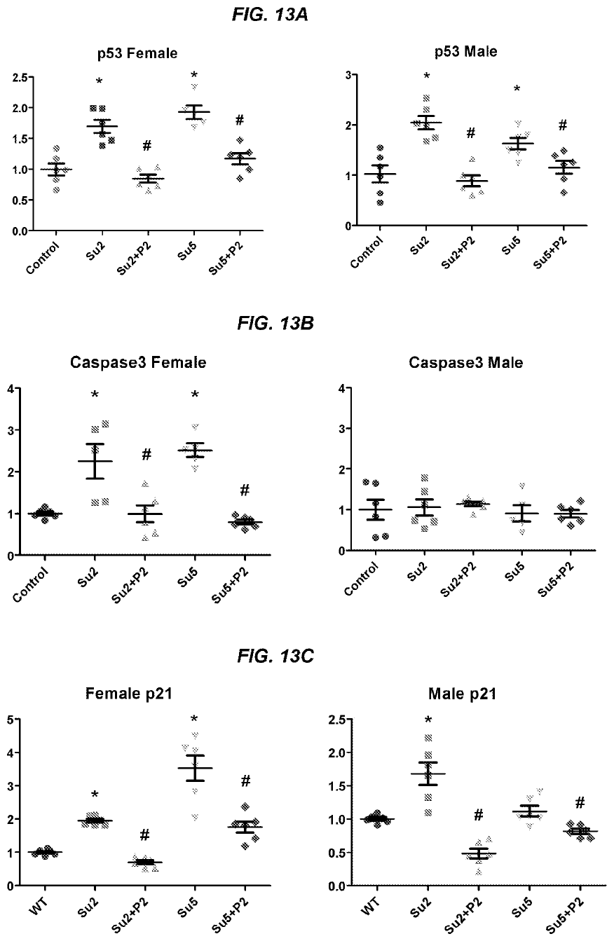

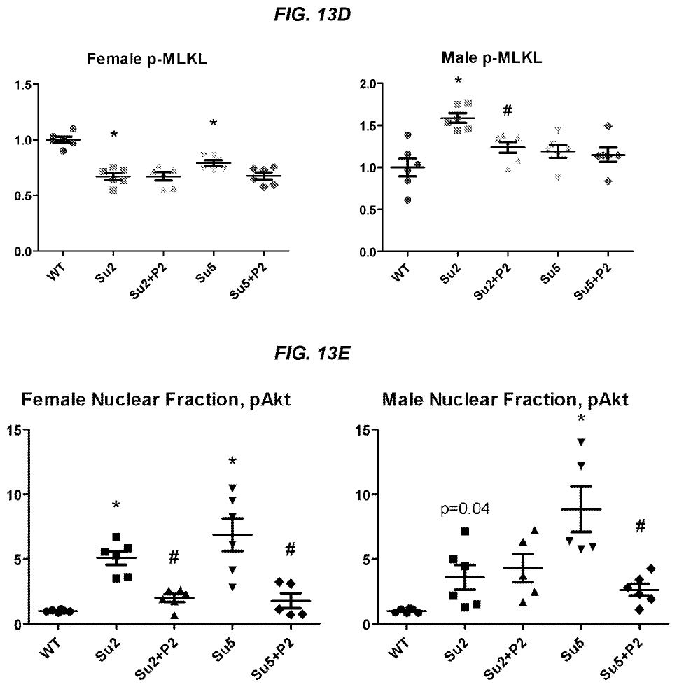

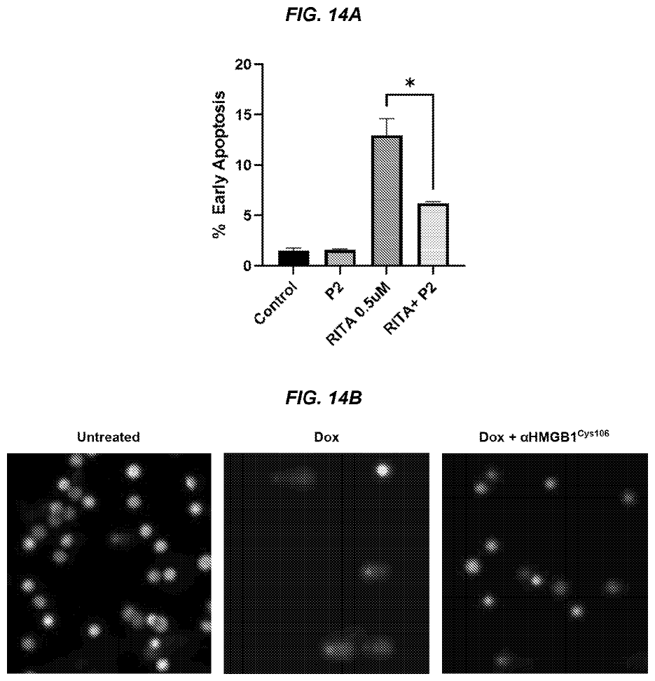

In some embodiments, the peptides described herein (e.g., FFLFCSEYRPKIKGRKKRRQRRRPQ (SEQ ID NO: 4)) may be anti-apoptotic (e.g., prevents apoptosis). In some embodiments, the peptides described herein (e.g., FFLFCSEYRPKIKGRKKRRQRRRPQ (SEQ ID NO: 4)) may be anti-apoptotic (e.g., prevents apoptosis) in female subjects (FIGS. 13B and 14A). In some embodiments, the peptides described herein (e.g., FFLFCSEYRPKIKGRKKRRQRRRPQ (SEQ ID NO: 4)) may be anti-senescent (e.g., prevents senescent), e.g., in male and female subjects (FIG. 13C). In some embodiments, the peptides described herein (e.g., FFLFCSEYRPKIKGRKKRRQRRRPQ (SEQ ID NO: 4)) may be anti-necroptotic (e.g., prevents necrosis). In some embodiments, the peptides described herein (e.g., FFLFCSEYRPKIKGRKKRRQRRRPQ (SEQ ID NO: 4)) may be anti-necroptotic (e.g., prevents necrosis) in male subjects (FIG. 13D). In some embodiments, the peptides described herein (e.g., FFLFCSEYRPKIKGRKKRRQRRRPQ (SEQ ID NO: 4)) may protect against genotoxic stress (FIG. 12E and FIG. 14B), e.g., in female and male subjects.

In some embodiments, the peptides described herein are anti-apoptotic, e.g., the peptide prevents apoptosis in female subjects. In some embodiments, the peptides described herein are anti-senescent, e.g., the peptide prevents senescence in male subjects and female subjects. In some embodiments, the peptides described herein are anti-necroptotic, e.g., the peptide prevents necrosis in male subjects. In some embodiments, the peptides described herein protect against genotoxic stress in female and male subjects.

### Example 1

The following is a non-limiting example of the present invention. It is to be understood that said example is not intended to limit the present invention in any way. Equivalents or substitutes are within the scope of the present invention.

To study the activities of HMGB1 cysteines, peptides were designed that bind to the specific regions of HMGB1 and, by shielding either Cys106 or Cys23/45, prevent their interaction with the downstream targets. The peptides with cell-penetrating TAT sequences were used to study the intracellular role of HMGB1 cysteines, while peptides without TAT served as extracellular controls. Fractionating pulmonary artery smooth muscle cells (PASMC) allowed the evaluation of HMGB1 cytosol/nuclear distribution. Calf thymus double-stranded DNA cellulose was used to study the binding of recombinant fully reduced HMGB1 in the presence or absence of a shielding peptide. The protective role of peptide in vivo was estimated using the rat Sugen/Hypoxia PH model. Given the discovered sex difference in the HMGB1-mediated signaling, the in vitro and in vivo studies were replicated in male and female animals and cells.

The cytosol and nuclear levels of HMGB1 were affected by a cell-penetrating peptide designed to shield Cys106 (TAT-α-HMGB1^{Cys106}, or α-Cys106) and not any other peptide. Further investigation revealed that α-Cys106 peptide produced ˜5.9 fold decrease in HMGB1 binding to DNA, enhanced the exit of nuclear HMGB1 to the cytosol, which was especially evident in male cells, and induced its proteasomal degradation (1±0.097 vs. 0.428±0.045 vs. 0.885±0.049 in untreated, α-Cys106 and α-Cys106/proteasome inhibitor (MG132, 10 μM, 1 hr) treated PASMC, p<0.001 and p<0.003, N=6). The peptide-mediated decrease in HMGB1/DNA binding negatively affected the expression of TLR4 and p53 and reduced cell apoptosis. In females, treatment with α-Cys106 (2.5 mg/kg/day, i.p during the wks 1-2 or wks 3-5) produced an anti-apoptotic effect and reduced plasma HMGB1 levels, which corresponded to a decreased right ventricle (RV) systolic pressure at the early stage of PH and protected the RV at the later stage. In contrast, male PH rats showed no apoptosis activation in the lungs, and α-Cys106 treatment produced no changes in plasma HMGB1 or hemodynamic parameters.

HMGB1^{Cys106} is the critical regulator of intracellular HMGB1 function, including HMGB1/DNA binding, regulation of p53 expression and apoptosis activation, the intracellular distribution of HMGB1, and its extracellular release. This study also confirms the significant sex difference in the levels of pulmonary apoptosis, HMGB1 release, and its contribution to the pathobiology of PH.

### Example 2

The following is a non-limiting example of the present invention. It is to be understood that said example is not intended to limit the present invention in any way. Equivalents or substitutes are within the scope of the present invention.

To study the activities of HMGB1 cysteines, peptides were designed with cell-penetrating TAT sequences that bind to either Cys106 or Cys23/45 and prevent the interaction of these cysteines with the downstream targets. The intracellular activities of peptides were evaluated by measuring HMGB1 cytosol/nuclear distribution and the ability to regulate the recombinant HMGB1 binding to double-stranded DNA. The protective role of the Cys106-shielding peptide (αHMGB1^{Cys106}; SEQ ID NO: 4) was estimated using the rat Sugen/Hypoxia (Su/Hx) PAH model and human endothelial pulmonary artery cells (hPAEC).

Only the αHMGB1^{Cys106} peptide affected the cytosol and nuclear levels of HMGB1 by inducing a 5.9-fold decrease in HMGB1 binding to DNA and enhancing the nuclear HMGB1 exit to the cytosol and lysosomal degradation. Treatment with αHMGB1^{Cys106} peptide (2.5 mg/kg/day, i.p for 2 wks) significantly attenuated PAH in Su/Hx-treated rats. In female rats, the right ventricle systolic pressure was significantly decreased in either preventive or treatment studies (Su/Hx vs. Su/Hx+αHMGB1Cys106: 73.2±3.4 vs. 50.4±3.0 mmHg, p=0.0002 and 110.5±6.3 vs. 70.0±2.8 mmHg, p=0.0005, N=6-8, correspondingly), while males were protected only by treatment protocol (101.7±2.7 vs. 73.4±6.4, p=0.0007, N=5-8). The molecular mechanism of αHMGB1^{Cys106} protection involved the negative regulation of p53 expression in either sex. However, the downstream effects of inhibited p53 signaling were found to be sex-specific. In females, attenuation of p53 expression was responsible for the reduced pulmonary apoptosis and senescence, while in males, the protection was associated with an inhibited genotoxic stress through the newly discovered nuclear p53/Akt pathway. The in vitro studies with male and female hPAEC confirmed the sex-specific role of p53 in apoptosis and genotoxicity and the therapeutic potential of the αHMGB1^{Cys106} peptide.

HMGB1^{Cys106} is critical in regulating HMGB1/DNA binding, p53 expression, and the sex-specific downstream p53 signaling, which includes apoptosis and senescence in females and genotoxicity in males.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only, and the claims are not limited by the dimensions of the figures. In some embodiments, descriptions of the inventions described herein using the phrase “comprising” includes embodiments that could be described as “consisting essentially of” or “consisting of,” and as such, meets the written description requirement for claiming one or more embodiments of the present invention using the phrase “consisting essentially of” or “consisting of”.

1. A peptide comprising a sequence at least 84% identical to FFLFCSEYRPKIK (SEQ ID NO: 1) or SIGDVAKKLGEMWNN (SEQ ID NO: 2), wherein the peptide binds to a high mobility group box 1 (HMGB1) protein, wherein the peptide comprises at least one modification.

2. (canceled)

3. The peptide of claim 1, wherein the modification is a substitution.

4. The peptide of claim 3, wherein the substitution comprises: at least one of the F amino acids is substituted with a Y or a W amino acid.

5. The peptide of claim 3, wherein the substitution comprises one or a combination of: the L amino acid is substituted with an I or a V amino acid, the C amino acid is substituted with an M amino acid, the S amino acid is substituted with a C or an M amino acid, the Y amino acid is substituted with an F or a W amino acid, the R amino acid is substituted with a K amino acid, the I amino acid is substituted with an L or a V amino acid, the A amino acid is substituted with a G amino acid, the D amino acid is substituted with an E amino acid, the E amino acid is substituted with a D amino acid, the V amino acid is substituted with an L or an I amino acid, or the M amino acid is substituted with an S or a C amino acid.

6.-9. (canceled)

10. The peptide of claim 3, wherein the substitution comprises one or a combination of: at least one of the K amino acids is substituted with an R amino acid, at least one of the G amino acids is substituted with an A amino acid, or at least one of the N amino acids is substituted with a Q amino acid.

11.-18. (canceled)

19. The peptide of claim 1, wherein the peptide is selected from a group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

20. The peptide of claim 1, further comprising a cell membrane crossing sequence attached to the peptide.

21. The peptide of claim 20, wherein the cell membrane crossing sequence comprises GRKKRRQRRRPQ (SEQ ID NO: 3).

22. The peptide of claim 20, wherein the peptide comprises a sequence at least 80% to identical FFLFCSEYRPKIKGRKKRRQRRRPQ (SEQ ID NO: 4) or SIGDVAKKLGEMWNNGRKKRRQRRRPQ (SEQ ID NO: 5).

23. The method of claim 20, wherein the cell membrane crossing sequence is modified, wherein the cell membrane crossing sequence is modified with a chemical group.

24. (canceled)

25. The method of claim 23, wherein the chemical group is a triphenylphosphonium group or derivative thereof.

26.-28. (canceled)

29. The peptide of claim 1, wherein the peptide binds at or around Cys106 or Cys23/45 of the HMGB1 protein.

30. The peptide of claim 1, wherein the peptide inhibits the HMGB1 interaction with DNA.

31. The peptide of claim 1, wherein the peptide is an anti-apoptotic peptide, an anti-senescent peptide, and/or an anti-necroptotic peptide.

32.-37. (canceled)

38. A method of treating an inflammatory disease in a subject in need thereof, the method comprising administering a therapeutically effective amount of a peptide according to claim 1 to the subject.

39. The method of claim 38, wherein the inflammatory disease is hypertension, systemic inflammation, acute or chronic heart disease, kidney disease, or liver disease.

40. A method of treating pulmonary arterial hypertension (PAH) in a subject in need thereof, the method comprising administering a therapeutically effective amount of a peptide according to claim 1 to the subject.

41. The method of claim 40, wherein the subject is female.

42. A method of inhibiting a high mobility group box 1 (HMGB1) protein interaction with DNA in vitro, the method comprising administering a peptide according to claim 1 to a cell or an in vitro system.

43. A method of inhibiting a high mobility group box 1 (HMGB1) protein interaction with DNA in a subject, the method comprising administering a peptide according to claim 1 to the subject.

44.-46. (canceled)
